(19) United States
(12) Patent Application Publication
(10) Pub. No.: US 2007/0135784 A1
(43) Pub. Date: Jun. 14, 2007

(54) Surgical drape with superabsorbent fluid management members

(75) Inventor: Terry N. Tankersley, Lagrange, GA (US)

Correspondence Address: DORITY & MANNING, P.A., POST OFFICE BOX 1449, GREENVILLE, SC, 29602-1449, US

(73) Assignee: KIMBERLY-CLARK WORLDWIDE, INC.

(21) Appl. No.: 11/301,598

(22) Filed: Dec. 13, 2005

(51) Int. Cl. A61B 19/00

(52) U.S. Cl. 604/357; 604/368

(57) ABSTRACT

A surgical drape for use during surgery on a patient includes a drape material sheet configured for covering at least a portion of the patient. Pocket structures containing a superabsorbent polymer (SAP) material are provided at discrete locations on the drape material sheet. The pocket structures include a liquid permeable top layer and a liquid impermeable bottom layer and are disposed to absorb fluids produced during a surgical procedure.

### BACKGROUND

Various configurations of disposable surgical drapes are well known in the art for keeping a surgical site on a patient sterile during a surgical procedure. A reinforcement area is often placed around a fenestration or an edge of disposable surgical drapes to provide structural strength and to absorb bodily fluids from the surgical site. Many disposable drapes also include a number of layers of different materials for the drape area and reinforcement area, with each layer providing a different property to the drape. For example, spunbond fabrics, meltblown fabrics, and polymer films have been used as layers in disposable drapes.

Certain surgical procedures involve large amounts of fluid, for example blood or saline irrigation fluid, at the point of surgery that must be absorbed or otherwise collected. Certain procedures also require the fluid to be removed from the point of surgery and safely contained within a container or absorbent material. For instance, towels or other absorbent material that is placed on the top surface of a surgical drape may be used to absorb this fluid. It is also the case that suctioning devices and surgical sponges are used to remove fluid that is within the patient during the surgical procedure.

Problems with the towels and other absorbent material placed around the point of surgery exist where the towel or absorbent material becomes so saturated with fluid that the fluid begins to wet the patient, clinician, and/or surgical table. As such, drapes have been provided with features that are designed to transport fluid away from the point of surgery to another point on the surgical drape where the fluid can be absorbed or removed. This is done in order to move the fluid from a zone proximate to the point of surgery to another location on the surgical drape that will reduce the likelihood of contamination to and from the patient and clinician. Drapes with a plastic trough attached to the surgical drape and positioned so as to transport fluid from the point of surgery to a more remote area of the surgical drape are examples of one way that such features are provided.

Also, these types of draining features have been made of the same absorbent material used in a surgical drape, but treated so as to be of lesser absorbency than the absorbent material into which the trough drains, for example by heat treating the particular portion of the absorbent material formed into the trough to make the trough less absorbent. However, these types of drainage features on surgical drapes are limited in that they are only capable of transferring fluid from one location on the drape to another location. Such features do not provide a means for readily absorbing relatively large amounts of fluid at the surgical site. Current drainage features on surgical drapes are only capable of transporting fluid from one location to another, and as such still allow a particular portion of the surgical drape to become saturated with fluid and hence increase the probability of the fluid leaking from the surgical drape and not being properly absorbed.

As such, a need currently exists for a surgical drape that has an increased capacity for absorbing and managing a relatively large volume of fluid produced during a surgical procedure.

### SUMMARY

Various features and advantages of the invention will be set forth in part in the following description, or may be obvious from the description, or may be learned from practice of the invention.

In accordance with aspects of the invention, a surgical drape is provided for use during surgery of a patient. The drape includes a material sheet having a size and configuration for covering at least a portion of the patient during the surgical procedure. In order to absorb and manage the flow of fluids that may be produced during the procedure, a superabsorbent polymer (SAP) material is incorporated with the drape material. The SAP is contained within a pocket structure that has a liquid permeable top layer and a liquid impermeable bottom layer at a desired location on the drape. Any number, shape, or configuration of pocket structures is contemplated within the scope and spirit of the invention. In a particular embodiment, the surgical drape includes a fenestration through which the surgical procedure is performed, and one or more of the pocket structures containing the SAP material is defined at least partially around the fenestration.

The pocket structure may be defined in various ways. For example, in one embodiment, the structure may incorporate at least one layer of the drape sheet material, and may be defined between opposed layers of the drape sheet material. For instance, the drape sheet material may include a liquid impermeable bottom film layer (i.e. a barrier layer) and a liquid permeable and absorbent top layer, with the pocket structure defined between these layers.

In an alternate embodiment, the pocket structure may be defined by at least one material that is attached to the drape sheet material. For example, the drape sheet material may be liquid permeable with the pocket structure including a liquid impermeable material attached to an underside of the drape sheet material, such as an adhesive film tape. The SAP material may be disposed between the sheet material and the attached liquid impermeable material.

To ensure that the SAP material does not migrate out from the pocket structure, it may be desired to seal the edges defining the structure. For instance, seal lines may be provided between the liquid permeable top layer and liquid impermeable bottom layer by any conventional bonding technique. The seal lines may be provided in a pattern corresponding to the desired location and shape of the pocket structures.

The SAP material may be provided in various forms. In one embodiment, the SAP material may be in particle form, or mixed homogeneously with a carrier material. In a particularly desirable embodiment, the SAP material is a coating applied to one of the materials forming the pocket structure. A commercially available SAP hot melt adhesive may be used for this purpose. The coating may be applied to one side of the liquid impermeable bottom layer of the pocket structure, such as a film tape, that is subsequently sealed to the underside of the permeable sheet material to define the pocket structure.

The pocket structures may also serve to define fluid control dams that channel fluid away from the surgical site, particularly after the SAP material has absorbed fluid and expanded causing the pocket structures to swell. In this regard, a plurality of the pocket structures may be disposed on the sheet material to define fluid control channels or trough zones between adjacent pocket structures. These trough zones may be used to channel fluid to a different area of the surgical drape, or to a fluid collection device, such as a pouch, that is attached to the drape. The troughs may be treated so as to be less absorbent than the liquid permeable top layer of the pocket structures. For example, the sheet material in the trough zones may be treated with a surfactant. In this manner, fluid is more readily channeled along the troughs and absorbed by the pocket structures.

As mentioned, any number of pocket structures may define any desired pattern on the drape. In a particular embodiment, the drape includes a fenestration with at least two pocket structures defined concentrically at least partially around the fenestration such that trough zones are defined around the fenestration between the pocket structures. The respective pocket structures may have the same or different amounts of the SAP material.

Many conventional surgical drapes include a reinforcement panel material disposed around a fenestration that includes an absorbent and liquid permeable material. This reinforcement panel may define the liquid permeable top layer of a pocket structure containing SAP material. In such an embodiment, the reinforcement panel may comprise sealed edges that also define a perimeter of the pocket structure. The pocket structure may encompass generally the entirety of the reinforcement panel, or a portion of the panel. For example, the pocket structure may be a border region around the perimeter of the reinforcement panel, or extend around the fenestration in the reinforcement panel.

In an embodiment wherein the reinforcement panel also includes a liquid impermeable bottom material, the pocket structure may be defined between the layers of the reinforcement panel.

In still an alternate embodiment, the pocket structures may be disposed between raised structures on the drape so as to define highly absorbent trough zones between the raised structures. The raised structure may be defined directly in the drape sheet material by conventional means, including molding, embossing, pleating, and the like, or may be defined by additional materials or structure attached to the drape sheet material. The raised structure may include a top layer that is rendered less permeable to fluids than the liquid permeable top layer of the pocket structures in the troughs.

Other features and aspects of the present invention are discussed in greater detail below by reference to exemplary embodiments depicted in the drawings.

### BRIEF DESCRIPTION OF THE DRAWINGS

FIG. 1A is a perspective view of a surgical drape incorporating aspects of the present invention.

FIG. 1B is a cross-sectional view of the layers of the drape taken along the line indicated in FIG. 1A.

FIG. 2A is a perspective view of an alternative drape embodiment.

FIG. 2B is a cross-sectional view of the drape taken along the line indicated in FIG. 2A.

FIG. 3A is a perspective view of still another drape embodiment according to the invention.

FIG. 3B is a cross-sectional view taken of the drape taken along the line indicated in FIG. 3A.

FIG. 4A is a perspective view of yet a different drape embodiment according to the invention.

FIG. 4B is a cross-sectional view taken of the drape taken along the line indicated in FIG. 4A.

FIG. 5 is a perspective view of an additional drape embodiment according to the invention.

FIG. 6A is a perspective view of still another drape embodiment according to the invention.

FIG. 6B is a cross-sectional view taken of the drape taken along the line indicated in FIG. 6A.

### DETAILED DESCRIPTION

Reference now will be made in detail to various embodiments of the invention, one or more examples of which are set forth below. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment, can be used on another embodiment to yield a still further embodiment. Thus, it is intended that the present invention cover such modifications and variations as come within the scope of the appended claims and their equivalents.

Surgical drapes formed in accordance with the present invention can generally possess any of a variety of sizes and shapes, depending on the particular use of the drape and on its desired properties. For example, certain surgical drape configurations are described in U.S. Pat. No. 6,055,987 to Griesbach, et al., which is incorporated herein in its entirety by reference thereto for all purposes.

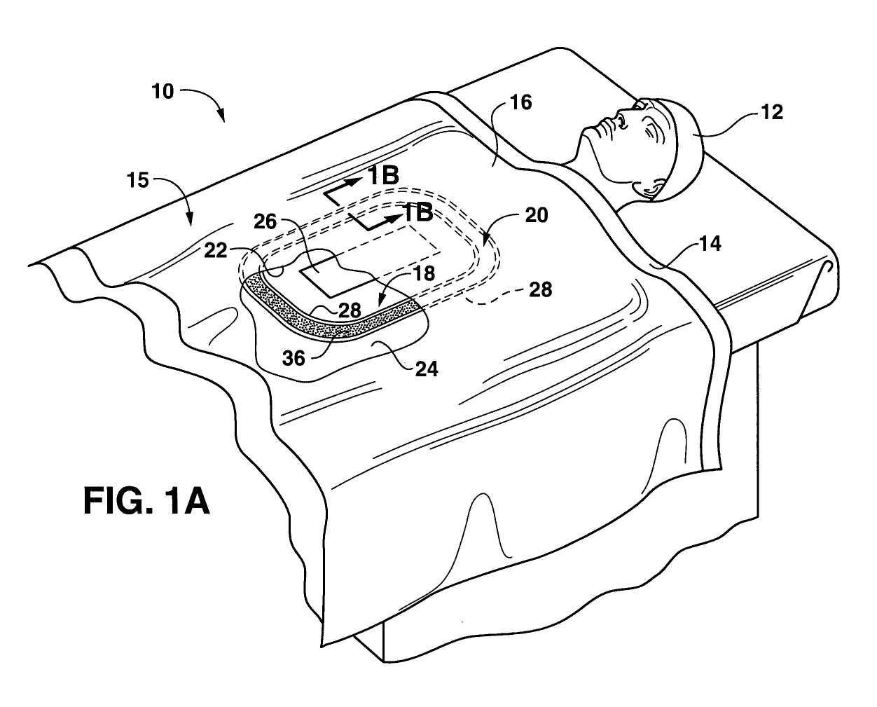

Various embodiments of surgical drapes incorporating aspects of the invention are depicted in the figures as drapes 10 for covering a patient 12 (FIG. 1) during a surgical procedure. The drapes 10 may be formed of any material or combination of materials defining a drape sheet material 15 commonly used in the art for disposable surgical drapes, garments, covers, and so forth. The types and properties of the material layers defining the drape sheet material 15 depends largely on the surgical procedure to be performed, and any combination of materials defining the drape sheet material 15, or intended use of the drapes 10, is within the scope and spirit of the invention.

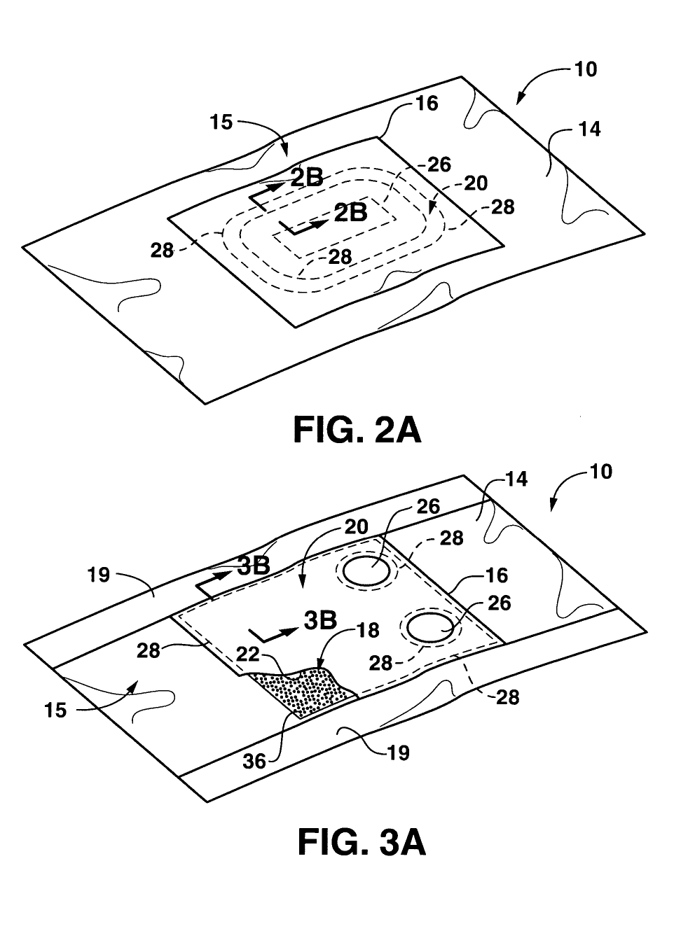

In general, the drape sheet material 15 typically includes a base material sheet 14. The base sheet 14 may be made from a wide variety of materials, including, for example, woven, reusable fabrics and nonwoven disposable fabrics or webs. Nonwoven materials suitable for use with the present invention include, for example, multilayer laminates such as a spunbond/meltblown/spunbond (“SMS”) material. An example of a suitable fabric is disclosed in U.S. Pat. No. 4,041,203, which is hereby incorporated by reference. The drape 10 may further include viewing panels 19 (FIG. 3A) made of a transparent, fluid resistant material, such as polyethylene film, for easy viewing and access to electrical table or c-arm controls and the like.

As used herein the term “nonwoven fabric or web” means a web having a structure of individual fibers or threads that are randomly interlaid, but not in an identifiable manner or pattern as in a knitted fabric. Nonwoven fabrics or webs have been formed from many processes such as for example, meltblowing processes, spunbonding processes, and bonded carded web processes. The basis weight of nonwoven fabrics is usually expressed in ounces of material per square yard (osy) or grams per square meter (gsm) and the fiber diameters are usually expressed in microns. (Note that to convert from osy to gsm, multiply osy by 33.91).

As used herein the term “spunbond fibers” or “spunbonded fibers” refers to small diameter fibers which are formed by extruding molten thermoplastic material as filaments from a plurality of fine, usually circular capillaries of a spinneret with the diameter of the extruded filaments then being rapidly reduced, for example, as in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartman, and U.S. Pat. No. 3,542,615 to Dobo et al. Spunbond fibers are generally not tacky when they are deposited onto a collecting surface. Spunbond fibers are generally continuous and have average diameters larger than 7 microns, more particularly, between about 10 and 20 microns.

As used herein the term “meltblown fibers” means fibers formed by extruding a molten thermoplastic material through a plurality of fine, usually circular, die capillaries as molten threads or filaments into converging high velocity, usually hot, gas (e.g. air) streams that attenuate the filaments of molten thermoplastic material to reduce their diameter, which may be to microfiber diameter. Thereafter, the meltblown fibers are carried by the high velocity gas stream and are deposited on a collecting surface to form a web of randomly disbursed meltblown fibers. Such a process is disclosed, for example, in U.S. Pat. No. 3,849,241 to Butin et al. Meltblown fibers are microfibers that may be continuous or discontinuous, are generally smaller than 10 microns in average diameter, and are generally tacky when deposited onto a collecting surface.

As used herein “multilayer laminate” means a laminate wherein some of the layers are spunbond and some meltblown such as a spunbond/meltblown/spunbond (SMS) laminate and others as disclosed in U.S. Pat. No. 4,041,203 to Brock et al., U.S. Pat. No. 5,169,706 to Collier, et al, U.S. Pat. No. 5,145,727 to Potts et al., U.S. Pat. No. 5,178,931 to Perkins et al. and U.S. Pat. No. 5,188,885 to Timmons et al. Such a laminate may be made by sequentially depositing onto a moving forming belt first a spunbond fabric layer, then a meltblown fabric layer and last another spunbond layer and then bonding the laminate in a manner described below. Alternatively, the fabric layers may be made individually, collected in rolls, and combined in a separate bonding step. Such fabrics usually have a basis weight of from about 0.1 to 12 osy (6 to 400 gsm), or more particularly from about 0.75 to about 3 osy. Multilayer laminates may also have various numbers of meltblown layers or multiple spunbond layers in many different configurations and may include other materials like films or coform materials, e.g. SMMS, SM, SFS, etc.

As used herein, the term “coform” means a process in which at least one meltblown diehead is arranged near a chute through which other materials are added to the web while it is forming. Such other materials may be pulp, superabsorbent particles, cellulose or staple fibers, for example. Coform processes are shown in commonly assigned U.S. Pat. No. 4,818,464 to Lau and U.S. Pat. No. 4,100,324 to Anderson et al. Webs produced by the coform process are generally referred to as coform materials.

In some embodiments, the drape 10 includes a fenestration opening 26 that can be placed over an operating site during surgery, as is well known in the art. The fenestrations 26 have a size, shape, and location that varies as a function of the particular type of surgical procedure the drape 10 is intended for. For example, drapes intended for use in femoral angiography procedures may include one or two generally circular fenestrations 26, as indicated in FIG. 3A. In alternate embodiments intended for thoracic procedures, the fenestration 26 may be generally rectangular, as illustrated in FIG. 1. The fenestrations 26 may be defined completely or partially through the drape sheet material 15.

A surgical drape 10 of the present invention may further include a reinforcement panel 16 superimposed on and affixed in any suitable and appropriate manner to the upper surface of base sheet 14. The width and length of the panel 16 may vary depending on the intended use of the drape 10. The reinforcement panel 16 may be formed from a variety of materials, such as a multilayer laminate that includes a fluid-absorbing material that may be backed by a fluid-repellent or fluid-impervious film layer. The film-layer side or lower surface of the panel 16 is secured to the upper surface of the base sheet 14 by any conventional means, including adhesive, stitching, thermal or ultrasonic bonding techniques. The absorbent upper surface the panel 16 remains exposed and available to absorb fluids emitted from the surgical site. The fluid-impervious film layer prevents the passage of blood and other body fluids through the reinforcement panel 16 and the base sheet 14. Any number of commercially available materials are suitable for use as the reinforcement panel 16.

In some embodiments, the upper surface of the reinforcement panel 16 may have an increased coefficient of friction to provide a slip-resistant surface to lessen the likelihood of undesired movement of surgical instruments that are placed upon the reinforcement panel 16. The coefficient of friction may be increased by providing a textured surface or by any other means known to those of skill in the art. The reinforcement panel 16 may be constructed of a material that has an absorbent upper surface to absorb fluids near the operative site. The reinforcement panel 16 also helps to inhibit penetration of the drape 10 by instruments that are placed on top of the reinforcement panel 16 during surgery.

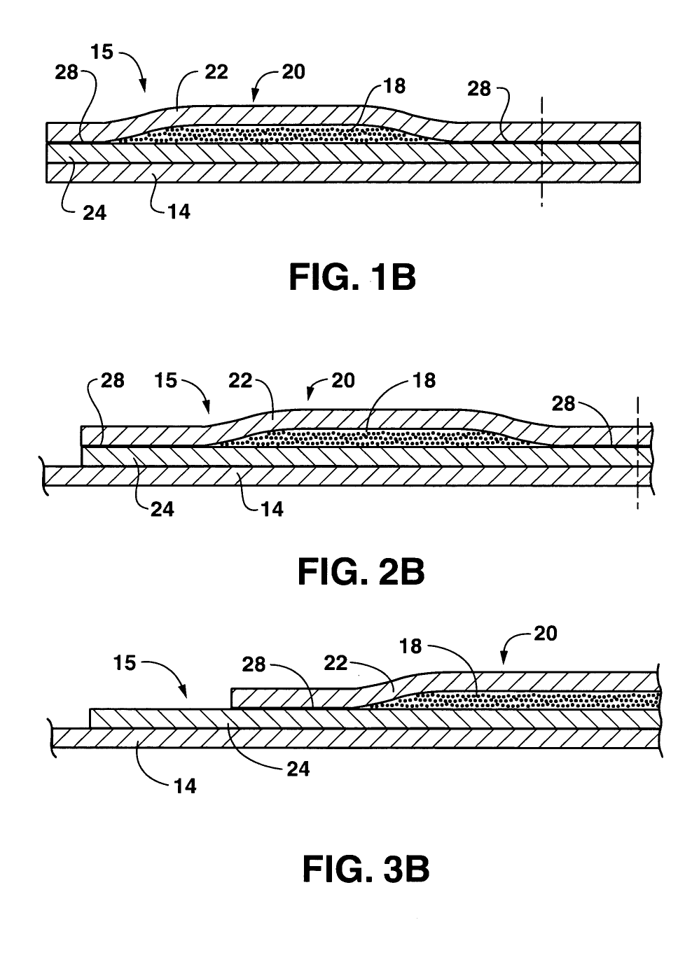

The reinforcement panel 16 may extend over a substantial portion of the base sheet 14, for example completely across the base sheet 14 as in FIG. 1A, or over a more limited region around the fenestration 26, as in FIG. 2A. The reinforcement panel 16 may include fenestrations that are aligned with the fenestration(s) 26 in the base sheet 14.

The reinforcement panel 16 may be a spunbond layer attached to a middle layer of a meltblown material, which is further attached to a fluid impervious film backing layer. This arrangement reinforces the area surrounding the fenestration 26 and also allows for fluid absorption. Construction of such a material 16 is described in U.S. Pat. No. 4,379,192 to Wahlquist et al. incorporated by reference herein in its entirety for all purposes.

As known in the art, an incise layer may be provided and positioned over the fenestration 26. This-incise layer may be formed from a low-density polyethylene film with adhesive on one side. For example, the incise layers may be constructed from polyethylene film available from Bertek Inc., St. Albans, Vt. 05478, or from a film available from Medical Concepts Development, Inc., St. Paul, Minn. 55125. In some embodiments, the incise layers may be constructed from an adhesive film available from 3M, Minneapolis, Minn. under the trade name 1525L. The incise layer may be disposed between the reinforcement panel 16 and the base sheet 14. The incise layers may include an adhesive side that is adapted to adhere to the patient when the drape 10 is placed over the patient.

In other embodiments, strips of adhesive (not shown) may be positioned around the periphery of the fenestration 26 to adhere the periphery of the fenestration to the patient. The tacky and pressure-sensitive adhesives used may be of any biologically acceptable adhesive. Examples of such adhesive materials are described in U.S. Pat. No. 3,669,106 entitled “Surgical Drape with Adhesive Attachment Means” to Schrading et al., which is incorporated herein in its entirety by reference.

Some or all of the materials used to form the drape sheet material 15 may be constructed so as to be hydrophilic or hydrophobic, and may be chemically treated to achieve the desired water absorbency properties. For instance, one or more materials may be treated with a surfactant in a manner such as described in U.S. Pat. No. 5,540,979, which is incorporated herein in its entirety by reference thereto for all purposes.

In certain embodiments, the drape sheet material 15 may include a bottom barrier layer, such as a barrier film made from 0.6 mil of polyolefin film, particularly for drapes 10 intended for surgical procedures that produce a large volume of fluids. A specific example of a barrier film is produced by Pliant Corporation and known as XP-928 Blue film.

Referring to the figures in general, drapes 10 according to the invention incorporate a superabsorbent polymer (SAP) material 18 incorporated within a pocket structure 20. The pocket structure 20 may have any shape or configuration, and any number of the pocket structures 20 may be utilized with a single drape 10. The pocket structures 20 containing the SAP material 18 are disposed so as to absorb and manage the flow of fluids that may be produced during a surgical procedure. For example, referring to FIG. 1, a pocket structure 20 may be defined essentially completely around the fenestration 26 to absorb and aid in the management of fluids produced during the procedure.

The pocket structures 20 include a liquid permeable top layer 22 and a liquid impermeable bottom layer 24. The SAP material 18 is contained between the top layer 22 and bottom layer 24. The top and bottom layers are sealed together at seal lines 28 to define the dimensions of the pocket structures 20. The seals 28 may be made by any conventional method for sealing the particular types of materials used as top and bottom layers 22, 24, such as adhesives, thermal bonding, ultrasonic bonding, and so forth. Any conventional bonding or sealing technique may be utilized to form a liquid impermeable seal between the top and bottom layers defining the pocket structures 20.

It should be appreciated that the pocket structures 20 may be defined by any combination of materials, including additional materials added to the base sheet 14, or materials that are incorporated as layers of the base sheet 14. Reference will be made herein to particular material arrangements, but it should be appreciated that the invention encompasses any configuration of materials that adequately define a pocket structure 20 having a liquid impermeable top layer 22 and impermeable bottom layer 24.

In the embodiment of FIGS. 1A and 1B, the pocket structure 20 is incorporated as a component of the fenestration reinforcement panel 16 that is attached to the base layer 14. In this embodiment, the pocket structure 20 is defined between a liquid impermeable top layer of the reinforcement panel 16 and a liquid impermeable bottom layer. The pocket structure 20 of this particular embodiment is defined by the spaced apart concentric seals 28 disposed completely around the fenestration 26. With this particular configuration, fluids produced at the surgical site will be absorbed at least partially by the upper layer of the reinforcement panel 16 and, as the fluid migrates out from the fenestration 26, will eventually come into contact with the SAP material 18 within the pocket structures 20. As is commonly known in the art, the SAP material 18 will readily absorb fluid and cause the pocket structure 20 to expand and thus define a dam-like structure around the fenestration 26. This structure serves to contain the outflow of fluids from the surgical site so that the fluids pool and have time to permeate through the top layer 22 of the pocket structure 20 for absorption by the SAP material 18.

SAP materials are well know to those skilled in the art, and any one or combination of readily available materials may be utilized with drapes 10 according to the present invention. Superabsorbent materials are water-swellable materials capable of absorbing at least about 20 times their own weight and, in some cases, at least about 30 times, in an aqueous solution containing 0.9 weight percent sodium chloride. The superabsorbent materials may be natural, synthetic or modified natural polymers and materials. Examples of synthetic superabsorbent material polymers include the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrrolidone), poly(vinylmorpholinone), poly(vinyl alcohol), and mixtures and copolymers thereof. Examples of natural or modified natural superabsorbent polymers include, for instance hydrolyzed acrylonitrile-grafted starch, acrylic acid grafted starch, methyl cellulose, chitosan, carboxymethyl cellulose, hydroxypropyl cellulose, and natural gums, such as alginates, xanthan gum, locust bean gum, and so forth. Mixtures of natural and wholly or partially synthetic superabsorbent polymers may also be useful in the present invention. Particularly suitable superabsorbent polymers are HYSORB 8800AD (BASF of Charlotte, N.C and FAVOR SXM 9300 (available from Degussa Superabsorber of Greensboro, N.C.).

The SAP material 18 may be provided in various forms within the pocket structures 20. In one particular embodiment, the SAP material may be in a loose, particulate form. The particles have a size so as not to migrate out through the permeable top layer 22 of the pocket structure 20. In an alternate embodiment, the SAP material may be mixed homogeneously with a carrier substance. For example, in a particularly desirable embodiment, the SAP material is applied as a coating 36 to one of the materials forming the pocket structure, for example to the impermeable bottom layer 22. In still an alternate embodiment, the coating may be applied to one side of a film strip or tape that is subsequently sealed to the underside of the permeable top layer 22 to define the pocket structure 20. In this embodiment, the impermeable bottom layer 24 is thus defined by the width of the tape structure. It may be further necessary to seal the edge regions of the tape along seal lines 28 to ensure that the SAP material does not migrate out from between the layers in use of the drape. An embodiment wherein the impermeable bottom layer 24 of the pocket structures 20 it is defined by a film strip or tape having a relatively limited width may be desired in that the pocket structures can be readily defined on drapes having a base sheet material 14 that is essentially liquid permeable (without a film barrier layer, or the like).

In a particularly useful embodiment, the SAP material 18 is applied in the form of an adhesive coating 36, wherein the SAP particles are homogeneously mixed with an adhesive material. Such a material is supplied under the trade name “Hydrolock” from H.B. Fuller Company, and is described in U.S. Pat. No. 6,534,572.

FIGS. 2A and 2B illustrate an embodiment of a drape 10 and incorporates a reinforcement panel 16 of significantly less surface area than the embodiment of FIGS. 1A and 1B. In this embodiment, a single pocket structure 20 is defined as a continuous race track structure around the fenestration 26 and, in this regard, is similar to the pocket structure 20 discussed above with respect to FIG. 1A. Pocket structure 20 is defined by the permeable top layer 22, which may be an upper layer of the reinforcement panel 16, and a liquid impermeable bottom layer 24, which may be a bottom layer of the reinforcement panel 16. In an alternate embodiment, the pocket structure 20 may be defined by additional materials placed upon the reinforcement panel 16.

FIGS. 3A and 3B illustrate an embodiment of a drape 10 configured as a femoral angiography drape containing circular fenestrations 26, as is well known in the art. This particular drape embodiment 10 also incorporates transparent side panels 19, such as a film material, attached along the longitudinal side edges of the base material 14. As can be particularly seen in FIG. 3A, the pocket structure 20 encompasses essentially the entire surface area of the reinforcement panel 16 except for the area of the fenestrations 26. Femoral angiography procedures are relatively fluid intensive, and this particular configuration may be desired in that it is capable of absorbing a significant volume of fluids generated from the operation. Seals 28 are defined generally around the perimeter of the reinforcement panel 16 and fenestrations 26, as particularly illustrated in FIG. 3A.

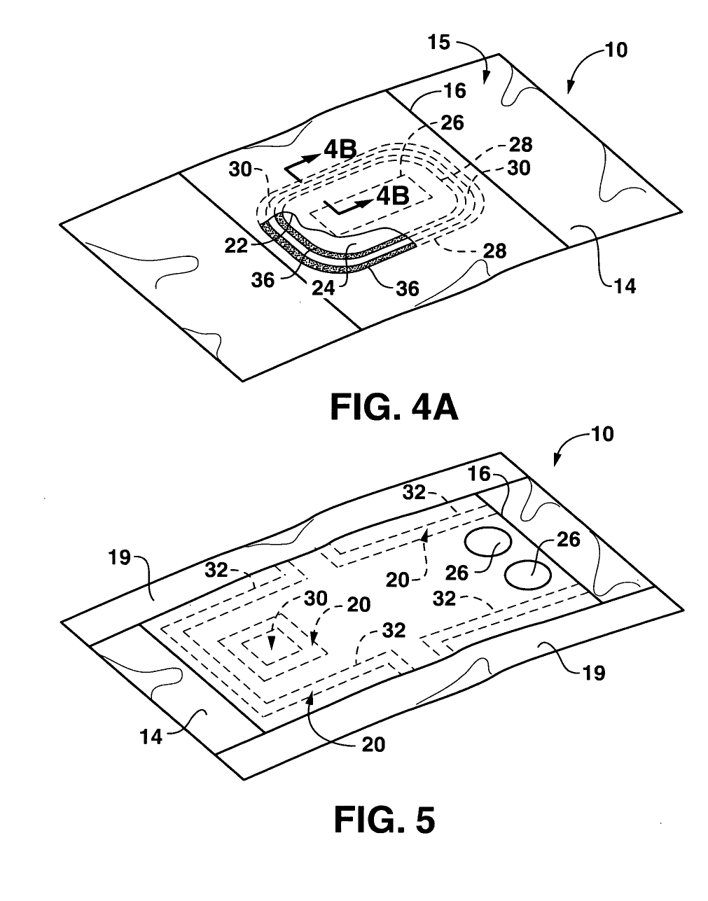

FIGS. 4A and 4B illustrate an embodiment of a drape 10 that includes at least two pocket structures 20 arranged concentrically with respect to the fenestration 26. The pocket structures 20 define fluid control channels or trough zones 30 between the structures, particularly when the SAP material 18 has absorbed fluid and swelled. These trough zones 30 may be used as storage regions for fluid between adjacent pocket structures until the fluid has had sufficient time to permeate through the top layer 22 for absorption by the SAP material 18. Referring to FIG. 4B, the trough zones 30 may be defined essentially by seal lines 28 between the adjacent pocket structures 20. In alternate embodiments, the pocket structures 20 may be spaced further apart, such that separate seals 28 are provided for each of the pocket structures 20.

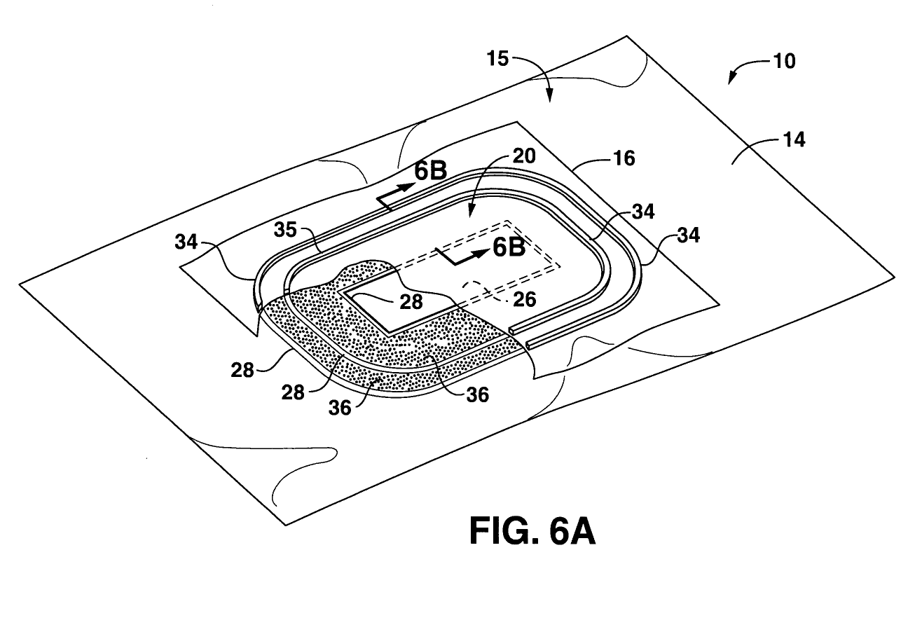

It should be appreciated that various structures and functions may be achieved by incorporating any number and pattern of the pocket structures 20. For example, referring to FIG. 5, a plurality of the pocket structures 20 are arranged to define channeling dams 32 at various portions of the upper surface of the drape 10. These dam structures 32 may be used to channel fluids to different areas of the surgical drape, such as to fluid collection bags or pouches attached at any desired position along the drape. The dams 32 may be used to partition off sections of the drape, particularly reinforcement panels 16, for placement or attachment of surgical tubing, devices, and so forth, to ensure that the attachment locations of such devices remain relatively free from exposure to fluids. It should be appreciated that any configuration and pattern of pocket structures 20 is within the scope and spirit of the invention.

The troughs 30 between or adjacent to pocket structures 20 may be treated to be less absorbent than the liquid permeable top layers 22 of the respective pocket structures 20. For example, the sheet material in the trough zones 30 may be treated with a surfactant to more readily channel fluids along the trials.

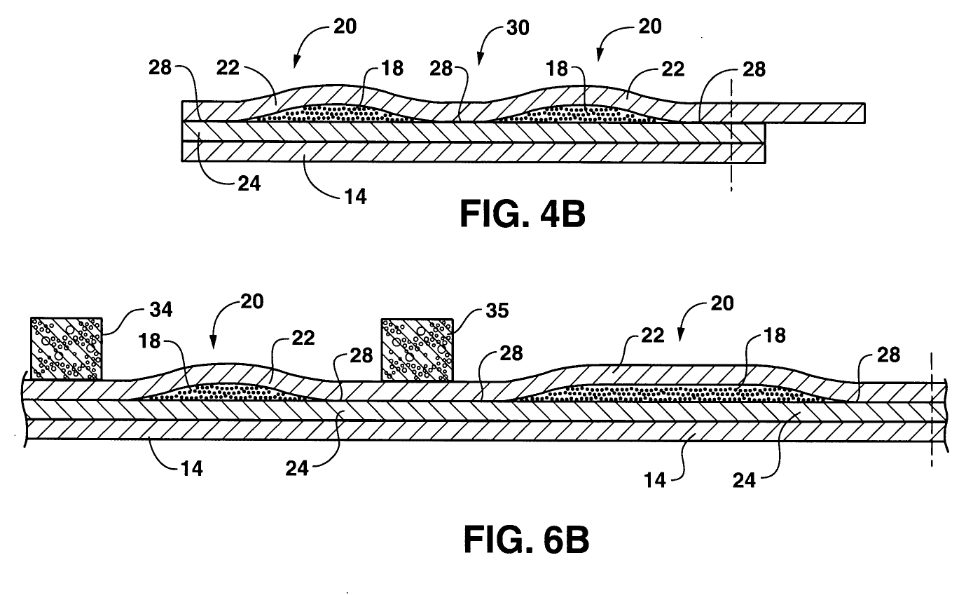

FIGS. 6A and 6B illustrate an embodiment of a drape 10 wherein raised structures 34 are provided on the upper surface of the drape material 15 around a pocket structure 20 containing SAP material 18. For example, referring to FIG. 6A, an outer raised structure band 34 completely encircles the perimeter of the pocket structure 20. An interior band 35 may also be provided. These raised structures 34, 35 may serve as fluid barriers or dams that retain fluids in the area of the pocket structure 20 so that the fluids may permeate through the top layer 22 for absorption by the SAP material 18. The raised structures 34, 35, essentially cause the fluids to pool within the area bordered by the structures. The raised structures 34, 35, may be a material, such as a foam or sponge strip, attached to the upper surface of the drape material 15, as illustrated in FIG. 6B. In an alternative embodiment, the raised structures may be defined in the sheet material by conventional means, including molding, embossing, pleating, and the like. The raised structures may be treated so as to be less permeable to fluids than the liquid permeable top layer of the pocket structures 20. It should be appreciated that any configuration or pattern of the raised structures 34, 35 is within the scope and spirit of the invention.

It should be appreciated by those skilled in the art that various modifications and variations can be made to the embodiments illustrated and described herein without departing from the scope and spirit of the invention. It is intended that the invention include such modifications and variations as come within the scope of the appended claims and their equivalents.

What is claimed is:

1. A surgical drape for use during surgery on a patient, comprising:
a drape material sheet configured for covering at least a portion of the patient during surgery; and
a superabsorbent polymer (SAP) material contained within a pocket 5 structure having a liquid permeable top layer and a liquid impermeable bottom layer at a desired location on said drape to absorb fluids produced during a surgical procedure.

2. The surgical drape of claim 1, further comprising a fenestration through which the surgical procedure is performed, said pocket structure defined at least partially around said fenestration.

3. The surgical drape of claim 1, wherein said pocket structure incorporates at least one layer of said drape sheet material.

4. The surgical drape of claim 3, wherein said pocket structure is defined by opposed layers of said drape sheet material.

5. The surgical drape of claim 1, wherein said pocket structure is comprises at least one material attached to said drape sheet material.

6. The surgical drape of claim 5, wherein said drape sheet material is liquid permeable, said pocket structure comprising a liquid impermeable material attached to an underside of said drape sheet material, said SAP material disposed between said sheet material and said liquid impermeable material.

7. The surgical drape of claim 1, wherein said pocket structure comprises sealed edges such that said SAP material does not migrate beyond said sealed edges in use of said drape.

8. The surgical drape of claim 1, wherein said SAP material comprises a coating applied to an inner face of said pocket structure.

9. The surgical drape of claim 8, wherein said coating comprises a hot melt adhesive containing SAP particles.

10. The surgical drape of claim 1, comprising at least two said pocket structures spaced apart with said drape sheet material between said pocket structures defining trough zones that are rendered less absorbent than said liquid permeable top layer of said pocket structure.

11. The surgical drape of claim 10, wherein said trough zones are treated with a surfactant.

12. The surgical drape of claim 10, further comprising a fenestration through which the surgical procedure is performed, and at least two said pocket structures defined concentrically at least partially around said fenestration, said trough zones defined around said fenestration.

13. The surgical drape of claim 2, wherein said drape sheet material further comprises a reinforcement panel material disposed around said fenestration, said pocket structure incorporated in said reinforcement panel.

14. The surgical drape of claim 13, wherein said reinforcement panel comprises sealed edges that also define a perimeter of said pocket structure.

15. The surgical drape of claim 14, wherein said pocket structure is defined as a border around said reinforcement panel.

16. The surgical drape of claim 14, wherein said pocket structure encompasses generally the entirety of said reinforcement panel.

17. The surgical drape of claim 1, wherein said pocket structure is disposed on said drape at a location so as to create fluid channeling dams in use of said drape to channel fluids from one region of said drape to another.

18. The surgical drape of claim 17, wherein said pocket structure is disposed so as to channel fluids to a fluid collection device attached to said drape.

19. The surgical drape of claim 1, further comprising raised structure on the surface of said sheet material defining fluid channeling troughs between said raised structure, said pocket structures defined in said fluid channeling troughs.

20. The surgical drape of claim 19, wherein said raised structure comprises a top layer that is rendered less permeable to fluids than said liquid permeable top layer of said pocket structures in said troughs.
